(19) United States
(12) Patent Application Publication
(10) Pub. No.: US 2008/0260851 A1
(43) Pub. Date: Oct. 23, 2008

(54) Polymeric Nanoparticles and Nanogels for Extraction and Release of Compounds

(75) Inventors: Ponisseril Somasundaran, Nyack, NY (US); Soma Chakraborty, Jersey City, NJ (US)

Correspondence Address: SCHWEGMAN, LUNDBERG & WOESSNER, P.A., P.O. BOX 2938, MINNEAPOLIS, MN, 55402, US

(73) Assignee: THE TRUSTEES OF COLUMBIA UNIVERSITY IN THE CITY OF NEW YORK, New York, NY (US)

(21) Appl. No.: 11/596,202

(22) PCT Filed: May 13, 2005
(86) PCT No.: PCT/US05/16912

§ 371 Date: Apr. 15, 2008

Related U.S. Application Data
(60) Provisional application No. 60/571,554, filed on May 13, 2004.

(51) Int. Cl. A61K 9/14; C08J 3/28; A61K 8/11; A61K 38/43; A61K 31/7052; A61K 31/13; A61K 31/445; A61K 38/16; A61K 39/395; A61Q 13/00

(52) U.S. Cl. 424/501; 522/153; 522/80; 522/79; 522/166; 424/130.1; 424/94.1; 514/12; 514/44; 522/87; 514/330; 514/656; 512/4

(57) ABSTRACT

The invention relates to polymeric nanoparticles and nanogels, which can contain, deliver, and/or release one or more active agents, such as biologically active molecules or fragrance molecules, and methods of preparing the polymeric nanoparticles and nanogels. The nanoparticles are crosslinked utilizing radiation (g-radiation) as the catalyst for free radical polymerization (see FIG. 1) rather than by toxic chemical means. The nanoparticles and nanogels can be modified, without limitation, with hydrophobic, hydrophilic, or ionic groups or moieties. or with enzymes. Methods of preparing nanoparticles and nanogels containing or encapsulating a variety of molecules, including biologically active molecules and fragrance molecules, are provided.

The U.S. Federal Government may have certain rights in this invention pursuant to National Science Foundation Contract/Grant Nos. 5-26738 and 5-24510.

This patent disclosure contains material that is subject to copyright protection. The copyright owner has no objection to the facsimile reproduction by anyone of the patent document or the patent disclosure, as it appears in the U.S. Patent and Trademark Office patent file or records, but otherwise reserves any and all copyright rights whatsoever.

### BACKGROUND OF THE INVENTION

Nanoparticles have many uses in the industrial, commercial, and medicinal arts. Nanoparticles can be constructed from polymeric materials, either naturally-occurring or synthetic. Nanoparticles can be crosslinked as well as modified or derivatized by conventional organic chemistry techniques to enhance their use in a variety of technologies.

There is a need in the art for new and improved nanoparticles, which are water dispersable, stable, and able to effectively incorporate, deliver or extract and release active molecules, substances, compounds, or ingredients at desired or needed sites and locations in both in vitro and in vivo environments. The present invention satisfies this need.

### SUMMARY OF THE INVENTION

The present invention provides polymeric nanoparticles and nanogels, which can be comprised of acrylic acid or acrylamide monomers. The nanoparticles and nanogels can be well-dispersed in water due to their size, and can contain, encapsulate, incorporate, extract and release molecules, such as active molecules, agents, or ingredients, including bioactive molecules, agents and ingredients. The active molecules, etc. can be released at a controlled rate over a long period of time. Methods of synthesizing the crosslinked, polymeric, poly(acrylic acid) or polyacrylamide nanoparticles and nanogels are encompassed by the invention. Further, methods for modifying these nanoparticles and nanogels with functional groups to yield their improved and useful properties are encompassed.

Methods of using the nanoparticles and nanogels to contain, encapsulate, incorporate, or extract different molecules, compounds, ingredients, or biological or chemical active agents are encompassed. Methods of using the nanoparticles and nanogels to deliver and release fragrances, or to deliver, extract and release bioactive agents, such as drugs and pharmaceuticals, are embraced by the present invention.

### DESCRIPTION OF THE FIGURES

FIG. 1 presents a representative schematic synthesis of poly((acrylic acid) nanoparticles using a reverse micro-emulsion technique.

FIG. 2 presents a Scanning Electron Micrograph (SEM) and enlargement of poly(acrylic acid) nanoparticles according to the present invention. The average particle size of the poly(acrylic acid) nanoparticles ranged from about 50 to 80 nm.

FIG. 3 presents effective diameters of the poly(acrylic acid) nanoparticles at neutral pH and alkaline pH.

FIG. 4 depicts and graphically demonstrates the extraction of linalyl acetate fragrance by poly(acrylic acid) nanoparticles.

FIG. 5 graphically presents the results of fluorescence probing of the microgel environment using pyrene.

FIG. 6 depicts the synthesis of a representative polyacrylamide microgel according to the invention.

FIG. 7 shows the characteristically narrow size distribution of 3% crosslinked microgels as described.

FIG. 8 shows that the size of microgel particles decreases with an increase in crosslinking.

FIG. 9 graphically presents fluorescence probing of a microgel environment using pyrene and a utility of the microgels as organic scavengers.

FIG. 10 represents a scheme for the synthesis of hydrophobically modified microgels.

FIG. 11 shows the solubility of hydrophobically modified microgel in water.

FIG. 12 graphically presents the release of fragrance, i.e., linalyl acetate (LA) from poly(acrylic acid) nanoparticles (PAANP) as a function of time at different pHs.

FIG. 13 shows a scanning electron micrograph of chiral poly(acrylic acid) nanoparticles synthesized by using 8 mol % of D-lysine.

FIG. 14 Shows CD and UV spectroscopy of L- and D-lysine PAANP in water.

FIG. 15 shows a scheme of synthesis of poly(acrylic acid) nanoparticles in the reverse microemulsion of Span 80 and Tween 80 in hexane.

FIG. 16 shows ¹H-NMR spectroscopy of poly(acrylic acid) nanoparticles in D₂O.

FIGS. 17A and 17B show effective hydrodynamic radius(Rh) of poly(acrylic acid) nanoparticles at pH 4 (FIG. 17A) and poly(acrylic acid) nanoparticles at pH 7 (FIG. 17B).

FIG. 18 shows the zeta potential of the nanoparticles as a function of the pH of the dispersion medium.

FIG. 19 shows a comparison of amitriptyline extraction by modified and unmodified poly(acrylamide) nanoparticles.

FIG. 20 shows a synthesis of chiral poly(acrylic acid) nanoparticles by cross-linking acrylic acid, lysine and N,N″methylene bisacrylamide inside the microemulsion of Span 80/Tween 80/hexane/water.

### DESCRIPTION OF THE INVENTION

The present invention embraces nanosized polymeric particles, also termed nanoparticles, for the containment, encapsulation, extraction, delivery and/or release of molecules or active agents, such as biological or chemical agents. Nonlimiting examples of such molecules or active agents include, without limitation, bioactive agents, pharmaceuticals, drugs, biocides, small molecules, chemicals, and fragrances.

Nanogels refer to types of spherical, covalently crosslinked, polymeric networks comprised of particles having particle sizes in the nanometer range. Nanoparticles embrace nanogels and nanocomposites, etc. Since nanogels have small size, porous structure, and the ability to be functionalized, they can serve as carriers for fragrances, drugs and other active molecules. In personal care industries, it is a crucial problem to incorporate water-incompatible materials or to trap molecules, such as perfume molecules, which rely on encapsulation to provide their unique attributes. Fragrances and other bioactive agents, e.g., those that function as antimicrobial agents, can be incorporated inside of the nanogels and can be well dispersed due to the sub micron size of the nanogel carrier. These incorporated molecules also remain protected or shielded from the surrounding environment. Fragrance molecules and antimicrobial agents trapped inside the nanogels can be released from the nanogels at a controlled rate for a prolonged period of time.

According to one embodiment of the present invention, nanogels can serve as effective drug delivery devices, or act as an antidote for the removal of overdosed drugs, e.g., by physical absorption, chemical interaction, or physicochemical absorption. As a result of their small size, nanogels are expected to pass through the capillaries without any obstruction. They can extract molecules, such as overdosed drugs, and be eliminated with minimal adverse effects on the human body.

Nanoparticles refer to a type of covalently cross-linked polymeric networks with a particle size in the nanometer range. Since nanoparticles are small size, porous, swellable and can be functionalized, they can act as efficient carriers for drugs, fragrances, biocides and other active molecules and as scavengers for toxins and overdosed drugs.

Nanoparticles can deliver therapeutic agents and proteins using various routes of administration due to their stability, homogeneity and better dispersion characteristics. These unique properties suggest their potential to scavenge and immobilize overdosed drugs/toxins present in the blood stream if properly designed for functionality, porosity and cleavage of weak linkages or the opening of surface gate. The invention encompasses poly(acrylamide) and poly(acrylic acid) nanoparticles useful for extraction of overdosed drugs. Drug toxicity in humans is one of the major health care problems, which can be induced by therapeutic miscalculation, illicit drug usage, or suicide attempt. For example, amitriptyline is an antidepressant drug and excessive use of amitriptyline is a suicide method in the United States. Similarly bupivacaine, which is used to provide anesthesia during the surgical procedure, if injected in excess amount causes cardiotoxicity. There is a need for scavenging systems for detoxifying overdosed patients by removing as much of the drug as possible within hours. Such systems should be either small enough or biodegradable in order to be excreted from the human body after the removal of the drug. The invention provides for synthesized poly(acrylamide) and poly(acrylic acid) nanoparticles useful to extract amitriptyline and bupivacaine.

Apart from using the using the nanoparticles as carrier of drugs and other pharmaceutical agents, they can also find applications in cosmetic, chemical and other industries if appropriately modified. From a design point of view, novel controlled release systems for consumer applications of this invention should have the following characteristics: (1) ability to release over a period ranging from minutes to days; (2) controlled (preferably constant) release rate; (3) selective adsorption/desorption, breakdown and open/close capacity; (4) use dry particles of micron or nanosize for better dispersion; (5) inertness; (6) non-toxic and non-carcinogenic properties; (7) a reasonable shelf life with stability under various transport and storage conditions.

There are several techniques available to prepare sub micrometer size polymeric nanoparticles. Of these techniques, the inverse microemulsion technique has been exploited to a very large extent. Free radical polymerization of monomers dissolved in these water-swollen micelles often results in monodisperse, spherical polymeric particles of size less than 1 mm. The challenges encountered in designing slow release systems in the cosmetic, pharmaceutical and chemical industries include: a) efficient dispersion of active ingredients, b) controlling the rate of extraction/release of the actives at desired time and place, c) enhancing the stability of the actives inside the nanoparticles, and c) improving the efficacy of extraction by modifying the nanoparticles. The invention addresses the above-mentioned issues.

The invention provides for polymeric nanoparticles for encapsulation of fragrances, antimicrobial agents, overdosed drugs and other actives. Neutral, cationic and anionic nanoparticles can be synthesized by microemulsion technique using gamma radiation. This method produces narrowly dispersed, spherical cross-linked polymeric networks. These nanoparticles can be further modified to introduce hydrophobic, hydrophilic, chiral and temperature sensitive moieties along the polymer backbone to increase their compatibility with the actives to be extracted or released. Fragrance encapsulation and their controlled release play a key role in fragrance marketing and cost savings. Fragrance samples attached to magazines and fliers as films or fine powders give consumers an opportunity to try the fragrance, which enhances its marketability. Encapsulation stabilizes the fragrance and controlled release prolongs the lifetime of the fragrance, thus effective in cost saving. Once the flavor or fragrance is encapsulated, controlled release can take place by diffusion, pressure gradient, temperature sensitivity and barrier/gate opening. The poly(acrylic acid) and modified poly(acrylic acid) substances of the invention can extract and release fragrances and flavors.

The invention incorporates cleavable linkages in the nanosystems so that after extraction of toxins or drugs, the weak linkages cleave making the particles small enough to be eliminated from the kidney. The invention provides for the synthesis of nucleic acid aptamers, synthetic oligonucleotides of modest size (˜15-100 nucleotides) that can bind to a particular ligand with great affinity and selectivity. Ligands can range from metal ions to small organic molecules to proteins to viruses and even to bacterial cells. Aptamers are created and selected using a combination of synthetic chemistry, enzymology and interfacial chemistry involving affinity chromatography.

Oligonucleotides not only have the ability to bind specific ligands, but in some cases can also catalyze a chemical reaction involving the ligand (most common reactions involve self-cleaving ability). In these cases the ligand becomes a substrate and the specific oligos are called DNAzymes. The invention provides for polyacrylamide nanoparticles crosslinked with these self-cleaving DNAzymes, which would act as triggers to sense the given ligand and deliver antidotes. For example, a DNAzyme specific to arsenic would cleave itself in its presence thus triggering the collapse of nanoparticles and release the embedded antidote. DNA aptamers and DNAzymes attached to nanoparticles may serve as sensors for toxins and microbes and carriers for the controlled release of antidotes. Preparation of these smart nanoparticles will require incorporation of the aptamers into the polymeric matrix of the nanoparticle structure. The invention provides for particles that have shells with gates or links that can be broken with the cleavable techniques. The invention provides for particles that are useful for extraction/release for particular sizes, porosity, shell thickness, functional groups, diffusion coefficient, electrostatic force, polarity (shell, interior), chirality, selectivity, solvent (e.g. pH), enzyme degradation and cleavage.

Once the nanoparticles are synthesized, extraction and release of actives into the nanoparticles can be triggered by the surrounding environment or by other external stimuli. As shown in Scheme 1, depending on the nature of the polymer, perturbations such as changes in temperature, pH or ionic strength can cause the system to shrink or swell. The resultant volume change causes extraction or release of the actives.

In one embodiment, poly(acrylic acid) nanoparticles (PAANP) and nanogels comprising acrylic acid monomers are encompassed by the invention. In another embodiment, poly(acrylamide) nanoparticles (PAMNP) and nanogels comprising acrylamide monomers are encompassed by the invention. Poly(acrylic acid) or polyacrylamide nanoparticles and nanogels can be utilized as vessels, vehicles, or carriers for drug and cosmetic molecules and ingredients, and as chemical reactors. Illustrative attributes of the PAANP and PAMNP of this invention include water solubility, nontoxicity, biocompatibility, pH sensitivity and bioadhesiveness. According to the invention, nanogels have narrow size distributions, can form stable suspensions in water, and can exhibit temperature and pH sensitivity. In an embodiment, polyacrylamide and poly(acrylic acid) nanogels having 5% crosslinking density can be synthesized; in such syntheses, the particle size increases with increasing temperature, wherein a size increase occurs at about 65° C. The size of nanogels can be systemically varied from about 50 to 90 nm by altering the crosslinking density. The ability to produce nanoparticles and nanogels of varying sizes allows for their use in the uptake or delivery of chemicals or other molecules, and in the extraction of toxins.

Without wishing to be bound by theory, the polymeric nanoparticles and nanogels of the present invention can swell or shrink under conditions or parameters of temperature, pH, light, etc. Swelling can enhance the pore size of the nanoparticles and nanogels; as a result, the mobility of entrapped, incorporated, or encapsulated molecules increases and more molecules can diffuse into the nanoparticles. If the polymeric network comprising the nanoparticles and nanogels shrinks, then the pore size decreases and those molecules that cannot be further incorporated into the pores are released.

In another embodiment, the nanogels and nanoparticles have an open network structure with large surface areas. With appropriate modification, i.e., chemical modification, these nanogels can be used as vehicles to extract pollutants or toxins, or as carriers for other substances, such as drugs or fragrances. In an embodiment, the polymeric nanoparticles and nanogels can be modified to incorporate one or more different functional groups or moieties, such as, e.g., one or more hydrophobic groups or moieties, one or more hydrophilic groups or moieties, one or more enzymes, one or more ionic or charged groups or moieties, and the like, or combinations thereof, for enhancing selectivity and/or specificity of the nanoparticles in different applications, e.g., substance release, detoxification, microreactors, etc. In some embodiments, all or a portion of the nanoparticle is modified, e.g., made hydrophobic, hydrophilic, or ionically charged. For example, the incorporation of hydrophobic chains into the nanoparticles and nanogels increases their hydrophobicity, thus allowing more efficient extraction of hydrophobic organic molecules, e.g., drugs and bioactive (or biologically active) agents. Modification of the nanoparticles and nanogels is performed by post grafting techniques in which nanoparticles are first synthesized by a reverse microemulsion process, which is followed by replacement of some of the functional groups in the nanoparticles by the desired modifying agents by conventional chemical reactions. (Examples 1 and 2). The choice of one or more modifying groups depends upon the type of molecule (or moiety) that is to be incorporated or encapsulated in the nanoparticles or nanogels, as well as upon the conditions under which the molecule (or moiety) is to be released. For example, for incorporation of the hydrophobic fragrance linalyl acetate into hydrophilic poly(acrylic acid) nanoparticles, the nanoparticles are modified with one or more hydrophobic moieties or groups, e.g., hexyl groups, to improve the efficiency of extraction/release.

In accordance with the invention, the polymeric nanoparticles and nanogels of the invention serve as excellent carriers of bioactive agents, as well as antidotes for the removal of drugs, toxins, poisons, and the like from the body, e.g., the removal of overdosed drugs from an animal's system, e.g., mammals, including humans. Nonlimiting examples of biologically active agents or molecules that can be contained within the nanoparticles and nanogels include drugs, neurochemicals, neuroleptics, peptides, proteins, chemotherapeutic agents small molecule pharmaceuticals, antimicrobial agents, antibiotics, antitoxins, detoxifying agents, antibodies, antifungal agents, enzymes, proteins, RNA molecules, antisense molecules, or a combination thereof. Combinations of two or more biologically active agents or molecules are also embraced by this invention.

In an embodiment, hydrophobic chains, e.g., hexyl groups, were introduced into nanogels to hydrophobically modify the nanogels. This modification is useful to extract and deliver organic molecules (e.g., drugs and fragrances) that have low solubility in water. In another embodiment, anionic charges were attached to the nanogels. The charged nanogels are used to absorb compounds having opposite ionic charges by electrostatic interactions. Both hydrophobic and charged nanogels showed significant extraction effects compared with the corresponding unmodified polyacrylamide and poly(acrylic acid) nanogels. In another embodiment, carboxylic acid groups were incorporated into the backbone of the nanogels to produce nanogels having an overall negative charge. Such modified nanogels show a significant increase in the extraction of selected target drugs, e.g., amitriptyline (antidepressant) and bupivacaine (anesthesia), compared with unmodified nanogels in water. In saline, the extraction efficiency of the charged nanogels decreased, while that of hydrophobic nanogels was higher.

As described in Example 1, the poly(acrylamide) (PAM) and poly(acrylic acid) (PAA) nanoparticles and nanogels of the present invention have a narrow size distribution and were synthesized by a reverse microemulsion polymerization technology involving gamma (γ)-radiation as the catalyst for free radical polymerization. The present method obviates the use of chemical catalysts, which are typically toxic and therefore require extensive manipulations and time to wash and render nontoxic. In contrast, γ-radiation polymerized nanoparticles and nanogels are virtually nontoxic compared with chemically catalyzed nanoparticles. Moreover, the risk of contaminating residual toxins from chemical synthetic procedures is overcome by the methods of making nanoparticles and nanogels according to the present invention. Thus, the nanoparticles and nanogels encompassed by this invention are more conducive to in vivo and animal use, including human use. Polymerized nanoparticles and nanogels prepared according to the invention are typically essentially spherical, monodispersed, water soluble nanoparticles having a size of about 10 to 100 nm, or about 50 to about 90 nm, or about 55 to about 85 nm. The size of the nanoparticles and nanogels can be controlled, for example, by varying the crosslinking density. The size and porosity of these materials is controlled by the number and length of the crosslinking agent added. For example, if the length of the crosslinking agent is kept constant, while the amount or numbers of crosslinking agent is increased, then the size and the porosity parameters of the nanoparticle and nanogel decrease. In addition, the size and the porosity parameters of the nanoparticle and nanogel decrease if the length of crosslinking agent is decreased, while the amount or numbers of crosslinking agent is kept constant.

For the preparation of hydrophobically-modified (i.e., hydrophobic) nanoparticles and nanogels, hydrophobicity is increased by increasing the chain length of the hydrophobic chains of introduced molecules and/or by increasing the ratio of the hydrophobic chains. Accordingly, the number of moieties contributing to hydrophobicity can be increased by increasing the length of the chain comprised of the hydrophobic units. Alternatively, by keeping the chain length short, the number of hydrophobic chains can be increased. Nanogels can be made more soluble by decreasing the crosslinking density and/or using fewer modifying hydrophobic groups or units. In a particular embodiment, N-acryloxysuccinimide was copolymerized into the poly(acrylamide), PAM, and the poly(acrylic acid), PAA, structures to prepare hydrophobic nanogels. This was followed by the substitution of succinimide by hydrophobic hexyl groups and propyl groups. In some embodiments, the efficiency of hydrophobically modified, or negatively-charged, poly(acrylamide) nanoparticles and nanogels to extract drugs, such as amitriptyline or bupivacaine, or other molecules, such as fragrance molecules, is increased relative to unmodified nanoparticles and nanogels.

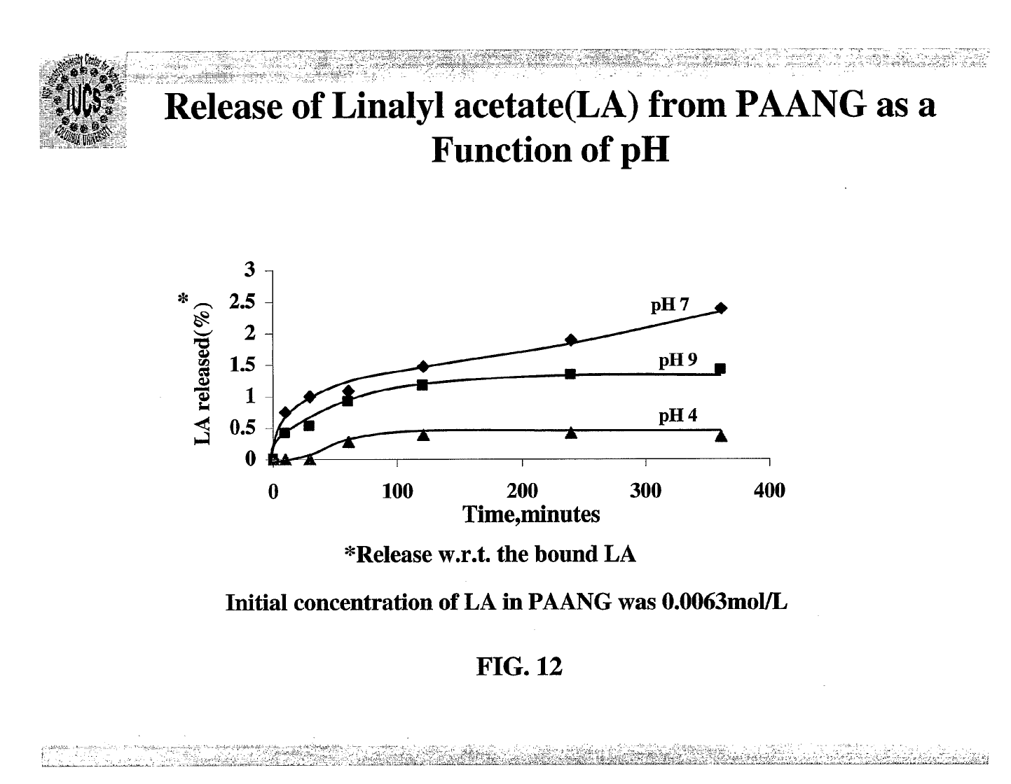

In another embodiment, fragrance molecules, or other active molecules, are incorporated and well-dispersed inside crosslinked poly(acrylic acid) nanoparticles and nanogels of the invention. The improved dispersion of encapsulated agents, e.g., fragrance, results from the sub-micron size of the carrier nanoparticles and nanogels. In an embodiment, the efficacy of fragrance extraction was enhanced when hydrophobically modified nanogels were employed compared with unmodified nanogels. Fragrance, e.g., linalyl acetate (lavender scent) vanillin, etc., and other agents can be released from the nanoparticles at a controlled rate for an extended period of time, e.g., up to about 4 hours and longer. The release rate for the molecules contained in the nanoparticles can be controlled as desired by changing the crosslinking density of the nanoparticles and the pH of the dispersion medium. For example, crosslinking density is changed by changing the amount of crosslinking agent used, or by changing the length of the crosslinking agent used. In an embodiment, the release of fragrance is dependent upon pH. For example, the amount of fragrance, e.g., linalyl acetate, released at alkaline pH was greater than that released at acidic pH, as determined by monitoring of the release profile of incorporated linalyl acetate as a function of pH of the dispersion medium. (FIG. 12). In another embodiment, unmodified poly(acrylic acid) nanogels at neutral or about neutral pH release more of their incorporated molecules, e.g., fragrance, compared with specifically-modified poly(acrylic acid) nanogels at neutral or about neutral pH. For example, unmodified and hexylamine-modified poly(acrylic acid) nanogels released about 2% of their encapsulated contents of linalyl acetate fragrance molecules at pH 7, while propylamine-modified poly(acrylic acid) nanogels released about 1% of encapsulated linalyl acetate fragrance at pH 7. Accordingly, the modification of nanogels can be tailored to the types of molecules that are encapsulated and the conditions under which the molecules are to be released.

Nonlimiting examples of other ingredients or molecules that can be encapsulated into and released from the nanoparticles and nanogels as described include fragrance molecules, dyes, colorants, UV absorbers, chemical compounds, drugs, pharmaceuticals, and small molecules of different types and function. Combinations of two or more encapsulated ingredients or molecules are also embraced by the invention.

The nanoparticles and nanogels of the present invention can be co-polymerized with temperature and pH sensitive monomers to obtain temperature and pH sensitive nanoparticles and nanogels whose release properties are temperature and pH dependent. Illustratively, nonlimiting examples of monomers that are suitable for producing temperature sensitive nanoparticles according to this invention include caprolactam and isopropylamide. In addition, with appropriate modifying groups, the nanoparticles and nanogels can be made light sensitive. For example, without limitation, photolabile groups can be incorporated into the nanoparticles and nanogels so that the molecules, e.g., fragrance or bioactive agents, contained therein are released upon exposure to light, e.g., ultraviolet (UV) or non-UV light.

The nanoparticles and nanogels of this invention are more uniform in size and are more stable for use as carriers for exogenous molecules, e.g., fragrance molecules, bioactive agents, and the like, because they are essentially solid materials. The solid nature of the nanoparticles described herein stands in contrast to non-solid or droplet particles known in the art. Also, the nanoparticles and nanogels of the invention are more porous, thus allowing for their better carrying/encapsulation capacity. The nanoparticles and nanogels of the invention can be employed in a variety of formulations, compositions, products, and the like, including, without limitation, pesticides; insect repellents; perfumes; other fragrances; cosmetics; toiletries; personal hygiene products; biocides; diapers; paper and plastic products, e.g., towels, napkins, tissue, storage and trash bags; pharmaceuticals; household products, e.g., air fresheners, fabric softeners, cleansers, cleaning agents, and detergents; soaps, deodorants, shampoos, moisturizers, body and facial creams, lotions and powders, etc.; clothing; bedding; linens, etc.

In other embodiments, the release of molecules contained within the nanoparticles and nanogels can be controlled, for example, by modifying their sensitivity to changes in temperature, pH, ionic strength, light, and the like. Nanoparticles and nanogels of the invention can be prepared to comprise other functional groups allowing for selective interaction with desired reactants, drugs, molecules, substances, pollutants, etc. Further, magnetic groups, e.g., iron oxide, can be attached to, or incorporated in, the nanoparticles and nanogels of this invention for manipulation of these materials, or their structure, using a magnetic field for the delivery, e.g., targeted delivery, and/or extraction of encapsulated molecules or substances.

When used in pharmaceutical formulation or compositions, a pharmaceutically- or physiologically-acceptable carrier, diluent, or excipient may be included with the nanoparticles and nanogels. Such carriers, diluents, or excipients include any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, disintegration agents, lubricants, sweetening agents, flavoring agents, dyes, such like materials and combinations thereof, as would be known to one of ordinary skill in the art (Remington's Pharmaceutical Sciences, 18th Ed., Mack Printing Company, 1990). Except insofar as any conventional carrier is incompatible with the active ingredient, its use in the therapeutic or pharmaceutical compositions is contemplated.

In an embodiment, modified poly(acrylamide) nanogels extract molecules, such as drugs, e.g., amitriptyline and bupivacaine, more efficiently compared with unmodified nanogels. In this embodiment, the efficiency of extraction increased markedly from 18% to 80%, as determined by using the Surface Plasmon Resonance (SPR) technique to evaluate the kinetics of extraction.

In a particular embodiment, the present invention encompasses a method of preparing polymerized nanoparticles, comprising: (a) solubilizing one or more nonionic or ionic surfactant, or a combination thereof, in organic solvent; (b) introducing one or more polymerizing reagent, e.g., poly(acrylic acid) monomers, polyacrylamide monomers, to the solution of step (a) to form a reaction mixture; (c) purging oxygen, e.g., atmospheric oxygen, from the reaction mixture, for example, under nitrogen gas; (d) exposing the reaction mixture of step (c) to gamma (γ) radiation for a time sufficient to crosslink nanoparticles formed in the reaction mixture; and (e) precipitating the crosslinked nanoparticles from the reaction mixture. In an embodiment, the organic solvent in the method comprises from 1-8 carbon atoms, which can be hexane. In another embodiment, the polymerizing reagent in the method comprises at least one of acrylic acid and bisacrylamide (e.g., N,N′-methylenebisacrylamide) monomers. In another embodiment of the method, the introducing step (b) further comprises a coupling or linking molecule or agent, which can be N-acryloxysuccinimide. In another embodiment of the method, the nanoparticles are precipitated with organic solvent, e.g., acetone. In another embodiment, the method further comprises washing the nanoparticles with organic solvent following step (d). In an embodiment, the organic solvent is hexane. In another embodiment, the method also comprises the step of modifying the nanoparticles and nanogels with a functional group, which can include one or more of hydrophobic groups, hydrophilic groups, enzymes, or ionic groups, as nonlimiting examples. In an embodiment, the functional group includes one or more alkylamine, such as, e.g., propylamine or hexylamine, or a combination thereof. In an embodiment, the method involves incorporating a fragrance molecule, e.g., ester-containing molecules, into the hydrophobically modified nanoparticles and nanogels. In another embodiment, the fragrance molecule is linalyl acetate or vanillin.

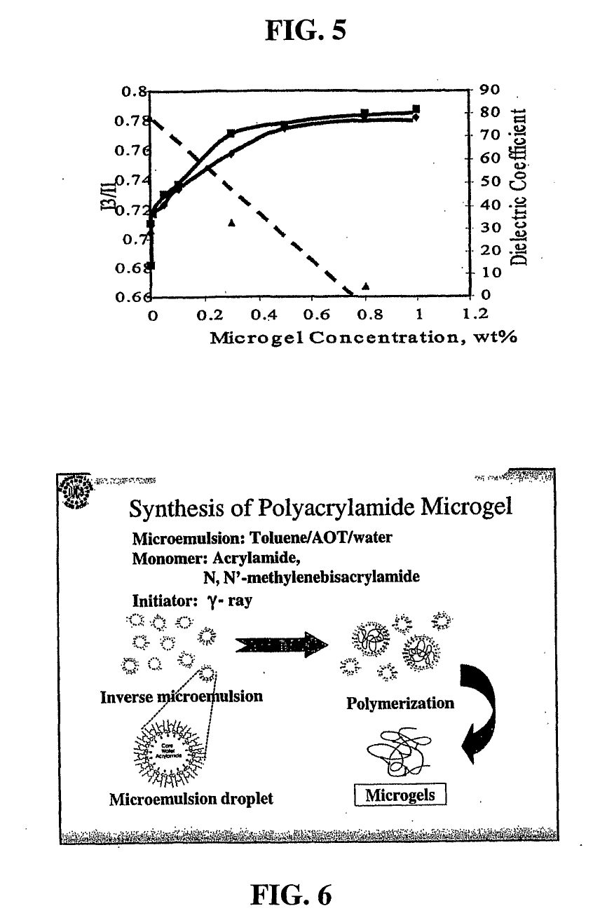

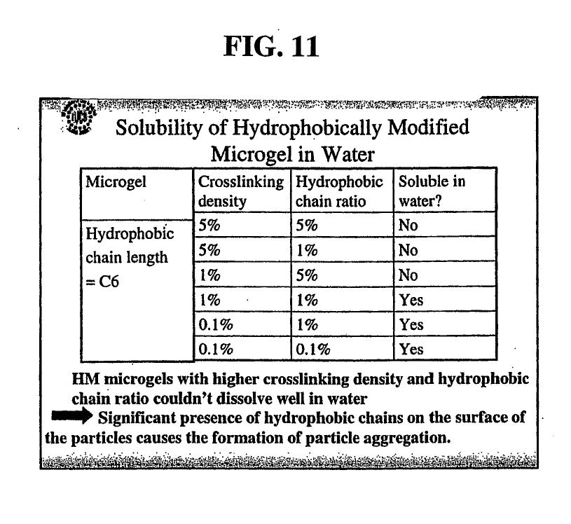

In other embodiments, the present invention relates to microgels as described in Example 5 and in FIGS. 5-11. In general, microparticles and microgels can be synthesized by methods similar to those used for nanoparticle and nanogel synthesis. Microgels can also be used for similar purposes. Nanoparticles and nanogels typically range in size from about 1 to 1000 nm, while the sizes of microparticles and microgels typically exceed this size range.

The following examples as set forth herein are meant to exemplify the various aspects of the present invention and are not intended to limit the invention in any way.

### EXAMPLE 1

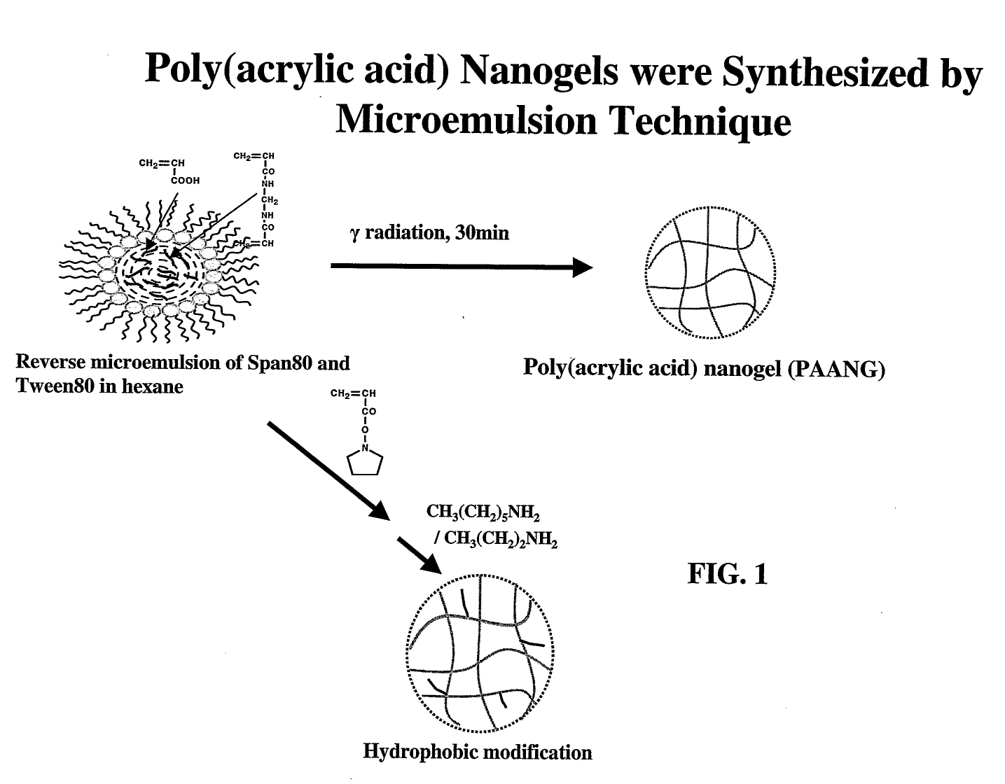

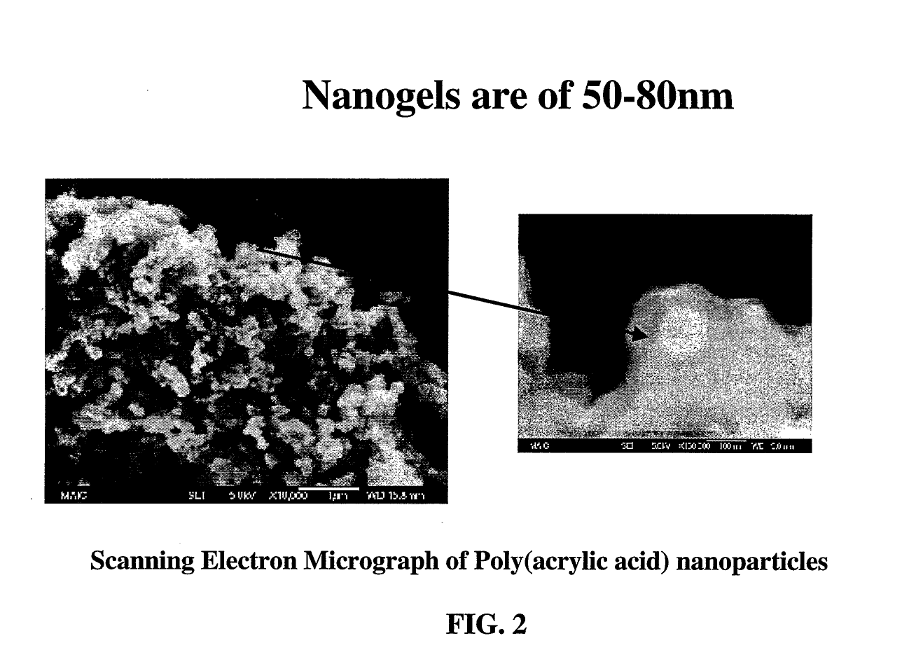

This Example describes the synthesis of representative poly(acrylic acid) nanoparticles according to the present invention. The surfactants sorbitan monooleate (SPAN 80®), (3.4 g), (i.e., anionic surfactant), and polyoxyethylene(20) sorbitan monooleate (TWEEN 80®), (2.6 g), (i.e., neutral surfactant), (Aldrich Co.) were solubilized in hexane (100 ml) to form a solution. To this solution, acrylic acid (0.8 ml, 0.016 mol) and N,N′-methylene bisacrylamide (0.0178 g, 0.0016 mol, in 1.3 ml of water) were added dropwise with stirring to form a reaction mixture. Thereafter, nitrogen gas was passed through the reaction mixture for 15 minutes to reduce or remove atmospheric oxygen. The reaction mixture was exposed to gamma (γ) radiation (600 rad/sec) for 30 minutes to obtain crosslinked poly(acrylic acid) nanoparticles. Acetone (20 ml) was added to the reaction vessel to precipitate the nanoparticles. The resulting nanoparticles were filtered and repeatedly washed with hexane to remove residual surfactant from the system. The method typically yielded nanoparticles having an average particle size of about 50-80 nm. (FIGS. 1 and 2). According to this example, polymerization occurs inside the microemulsion of the SPAN 80®/TWEEN 80®/hexane/water solution by γ irradiation. It will be appreciated that variation in the ratio of surfactant:organic solvent:water in the method can adversely affect the stability of the resulting microemulsion.

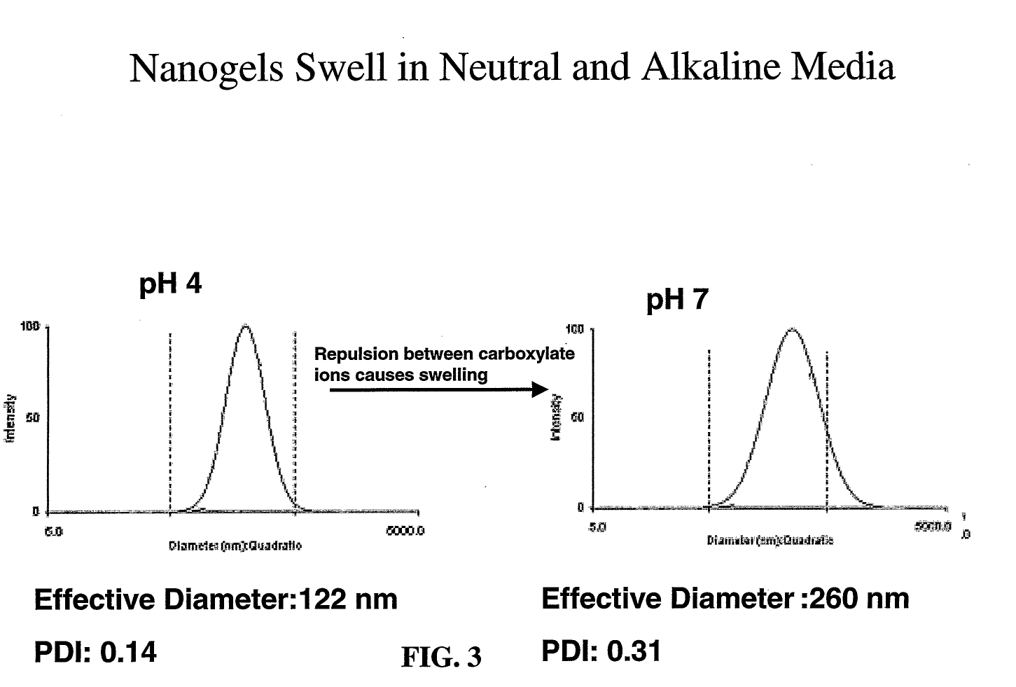

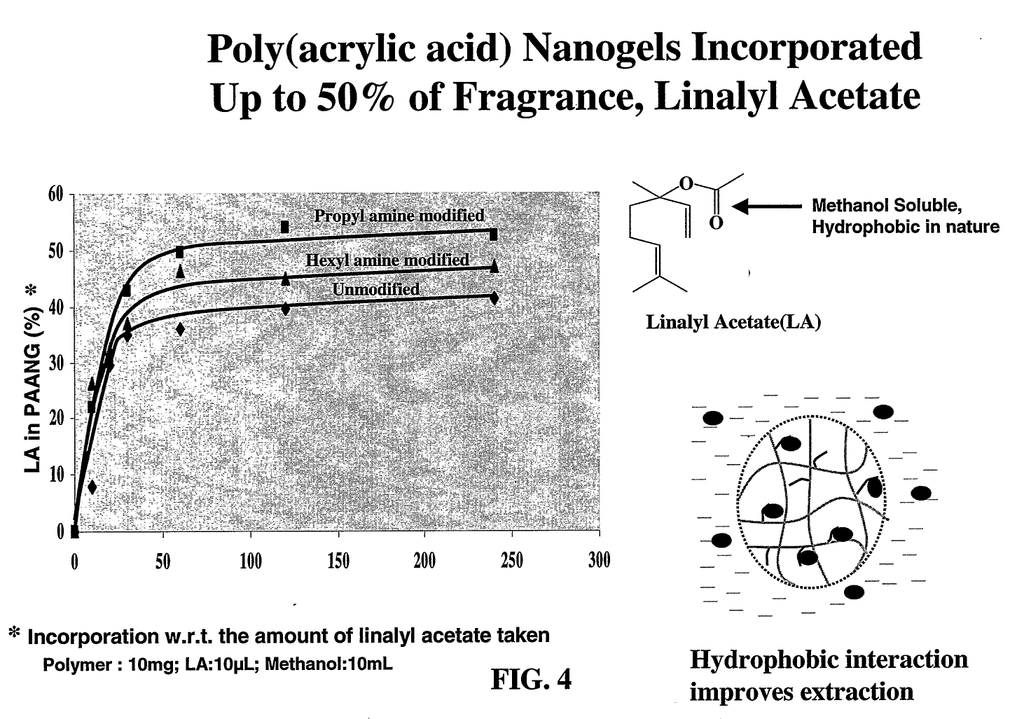

Zeta potential measurement showed that the prepared nanogels were negatively charged as a result of the presence of the negatively charged carboxylate ions. The negative value of the zeta potential increased with change in pH of the dispersion medium from pH 2 to pH 7. Further increase of pH of the dispersion medium from pH 7 to pH 12 decreased the negative zeta potential value. In addition, the effective hydrodynamic radius of the particles, as determined by dynamic light scattering analysis, showed that under neutral and alkaline conditions, the nanogels synthesized as described herein swelled to nearly four times their original dimension (FIG. 3).

### EXAMPLE 2

This Example describes the synthesis of hydrophobically modified poly(acrylic acid) nanoparticles according to this invention. (FIG. 1). The surfactants SPAN 80® (3.4 g) and TWEEN 80® (2.6 g) were solubilized in hexane (100 ml) to form a solution. To this solution, acrylic acid (0.8 ml, 0.016 mol), and (N,N′-methylenebisacrylamide (0.0178 g, 0.0016 mol) and N-acryloxysuccinimide (0.0196 g, 0.0016 mol), both dissolved in 1.3 ml of water) were added dropwise with stirring to form a reaction mixture. Thereafter, nitrogen gas was passed through the reaction mixture for 15 minutes. The reaction mixture was exposed to gamma (γ) radiation (600 rad/sec) for 30 minutes to obtain crosslinked poly(acrylic acid) nanoparticles. Acetone (20 ml) was added to the reaction vessel to precipitate the nanoparticles. The resulting nanoparticles were filtered and repeatedly washed with hexane to remove residual surfactant from the system. To the 10 ml of aqueous dispersion of nanoparticles (0.1 g/ml), dimethylfluoride (DMF), (10 ml), containing 0.0016 mol of alkyl amine, propylamine and hexylamine, were added dropwise, and the reaction mixture was stirred for 12 hours at 25° C. The product was precipitated in acetone.

### EXAMPLE 3

This Example describes hydrophobically modified poly(acrylic acid) nanoparticles having a fragrance, i.e., linalyl acetate, incorporated therein and the release of the fragrance by the resulting nanoparticles. The hydrophobically modified (poly(acrylic acid) nanoparticles, (10 mg), i.e., PAANP, as described in Example 2 were dispersed in methanol (10 ml) to which 10 μl of linalyl acetate were added to form a reaction mixture. The reaction mixture was stirred at 25° C. Aliquots removed from the reaction mixture at different time intervals were diluted 10 times in methanol and filtered through a 0.2 μm filter. The concentration of the residual linalyl acetate was determined by UV spectroscopy.

The linalyl acetate-incorporated PAA nanoparticles were recovered from the reaction mixture by centrifugation and the product was washed with methanol. The resulting nanoparticles were dispersed in water. The concentration of the linalyl acetate in water at different time intervals over a four hour period was determined by UV analysis of aliquots of the fragrance-containing nanoparticles. Fragrance was found to be released for four hours and release continued beyond four hours. Fragrance release was also observed to be pH dependent, e.g., more fragrance was extracted at a neutral pH (pH 7) and alkaline pH (pH 9) than at an acidic pH (pH 4). (FIG. 12).

### EXAMPLE 4

This Example describes the extraction of the drugs bupivacaine and amitriptyline by poly(acrylic acid) nanogels. These drugs are responsible for cardiotoxicity, if administered in excess quantity. To monitor the extraction of drug, 10 mg of the nanogel was dispersed in 10 ml of buffer (pH 4, pH 7, and pH 9) followed by the addition of 1.0 mg of the drug. The reaction mixture was stirred at 25° C., aliquots were removed at different time intervals and the residual concentration of the drug in the aliquots was determined by UV analysis. It was observed that extraction was pH dependent. Poly(acrylic acid) nanogels extracted around 60% of bupivacaine and amitriptyline in 4 hours at pH 7 and pH 9. At pH 4, however, the extraction of both bupivacaine and amitriptyline was negligible. Moreover, at pH 7 and 9, the amount of amitriptyline extracted was greater compared with the amount of bupivacaine.

### EXAMPLE 5

This Example describes the synthesis of a polyacrylamide microgel by an inverse microemulsion polymerization procedure. (FIG. 6). The inverse microemulsion comprised a water/oil system, e.g., water/toluene, that was stabilized by the surfactant sodium bis 2-ethylhexyl sulfosuccinate (Aerosol-OT or AOT), (Fluka Chemicals). The water soluble monomers (acrylamide and N,N′-bismethyleneacrylamide) were dissolved in the water droplets in the reverse microemulsion and irradiated by γ-radiation. The size of the crosslinked polymeric particles was effectively controlled by the size of the inverse emulsion droplets, by controlling the ratio of oil, surfactant and water in the system.

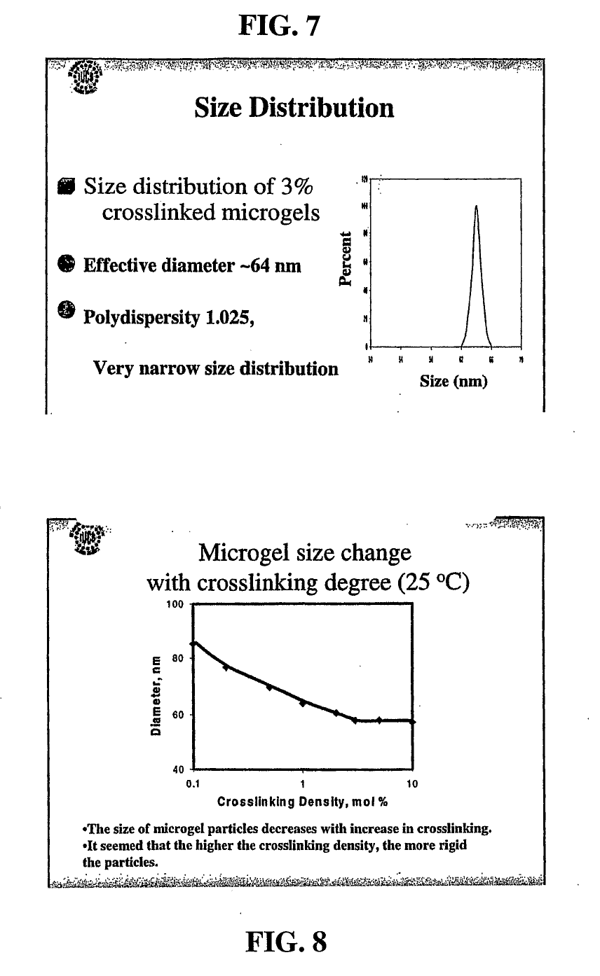

Particle size was determined by dynamic laser light scattering. The microgel particles resulting from this preparation scheme are monodispersed; the size of the particles changed from 55 nm to 85 nm as the crosslinking density decreased from 10% to 0.1%. (e.g., FIGS. 7 and 8). This effect can be used to adjust the porosity of the particles, since a decrease in the crosslink density results in more porous particles. Similar results were obtained from scanning electron microscopic (SEM) examination. Microgels were confirmed to be spherical particles with a very narrow size distribution.

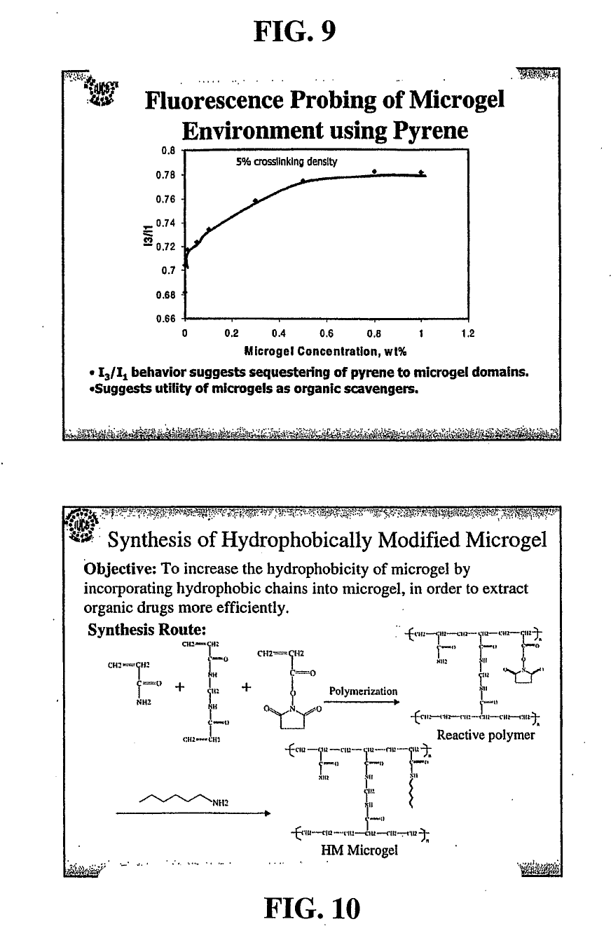

Pyrene was used as a probe to monitor the hydrophobicity of the microgels. (FIGS. 5 and 9). At constant pyrene dosage, the polarity parameter (I₃/I₁), where I₃ and I₁ are the third and first vibronic peaks in the fluorescence spectrum, respectively), were found to increase with increase in microgel concentration, suggesting that the microgel interior is somewhat more hydrophobic than the exterior environment. (An I₃ I₁ value of 0.6 corresponds to aqueous environment, and a value of 0.85 corresponds to the environment of a micellar interior). (FIG. 5). It was observed that the solubility of pyrene increased with the concentration of the microgels, thus demonstrating that microgels have useful solubilization capacity for organic molecules. The Ie/Im, i.e., the ratio of excimer peak to the monomer peak, could be observed in the fluorescence spectra, which also demonstrated that the local concentration of pyrene increased measurably.

To evaluate the interaction force between/among the particles, the viscosity of dilute and concentrated microgel suspensions was measured. In the dilute concentration range, the interactions between the particles were very weak, suggesting that the particles were almost neutral in suspension.

Water soluble microgels containing hydrophobic domains that can extract and immobilize hydrophobic compounds, substances, or molecules were prepared. Acrylamide (1.75 g), N,N′-methylenebisacrylamide (0.19 g in 5.0 mL distilled water) and N-acryloxysuccinimide monomers were polymerized in an AOT/H₂O/toluene (AOT (8.75 g) in 34.5 g of toluene) microemulsions system. N-hexylamine was grafted to the microgel and then purified by precipitation, filtration and dialysis. In the first series of tests, 5% of hydrophobic chains were introduced into a 1% crosslinked microgel sample.

The presence of significant amounts of hydrophobic chains on the surface of the hydrophobically modified microgels results in a decreased water solubility of the modified microgels. To overcome this problem, the ratio of hydrophobic chains can be decreased from 1% to 0.1%, and the crosslinking density can be decreased from 1% to 0.1%, for example. Thus, fewer hydrophobic groups remain on the surface, and the openness of the gel is increased to permit more chains to enter the interior of the gel. Also, less hydrophobic amines can be used. Less hydrophobic amines can be first adsorbed on the surface as a “sacrificial” agent and thus prevent the more hydrophobic chains from reacting with the active sites on the surfaces (N-acryloxysuccinimide is the reactive site).

### EXAMPLE 6

### D/L-Lysine Induced Chirality in Poly(Acrylic Acid) Nanoparticles

The use of circular dichroism is examined as a probe for detection of chiral lysine molecules embedded in poly(acrylic acid) nanoparticles (PAANP), giving rise to macromolecular structures with a helical twist correlating with the chiral center of chiral L- and D-lysine. L- and D-lysine are able to induce a circular dichroism (CD) signal in achiral PAANP.

The materials trapped inside the nanoparticles can be released at a controlled rate at the target site. They are generally inert, with a reasonable shelf life and can be dispersed well in various types of formulations. The stability of the encapsulated substances is superior inside the core of nanoparticles and the guests remain protected against a hostile environment. Conformational studies of the nanoparticles composed of diblock copolymers of poly(γ-benzyl L-glutamate)(PBLG) and poly(ethylene oxide)(PEO) carried out by circular dichroism (CD) showed that the nanoparticles have a α-helical conformation. The PBLG homopolymer has a right-handed helical sense and gets inverted to left-handed helix with the incorporation of POE.

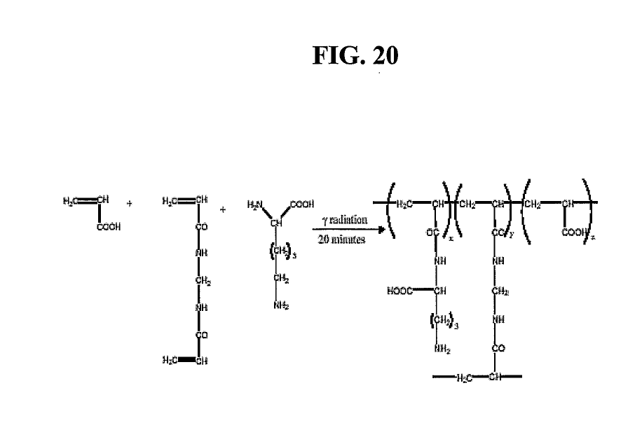

The nanoparticles may be used as devices to deliver optically pure drugs and enantioselectively encapsulate flavor or fragrance ingredients. The synthesis of the nanoparticles involves copolymerization of acrylic acid with D- and L-lysine by reverse microemulsion technique as shown in Scheme 1 in FIG. 20. Scanning electron micrograph (SEM) revealed the particles to be 100 nm. Optical activity induced in the nanosystem by lysine was detected by CD measurements.

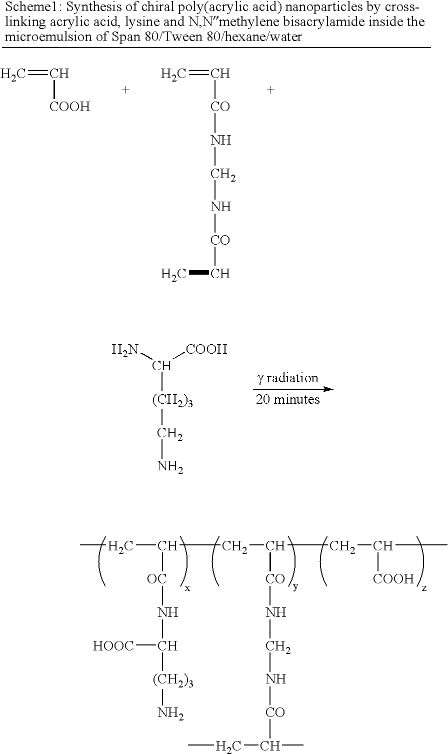

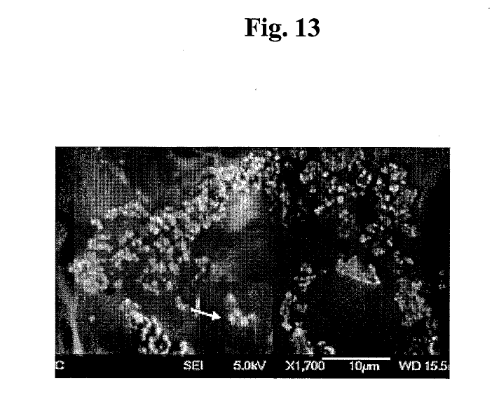

As an example of a typical procedure, the synthesis of chiral poly(acrylic acid) nanoparticles with 5-mol % of L-lysine was performed as follows. 3.4 gm of Span 80 and 2.6 gm of Tween 80 were added dropwise under vigorous stirring to 100 mL of hexane in a round bottom flask capped with a rubber septum. 0.8 ml (0.84 g, 1.16×10⁻² mol) of acrylic acid was added to the round bottom flask followed by the dropwise addition of 1.3 mL aqueous solution of the cross-linker, N,N.-methylenebisacrylamide (0.1 g, 8.8×10⁻⁴ mol) and L-lysine (0.085 g, 5.8×10⁻⁴ mol, 5 mol % of acrylic acid) under vigorous stirring. Throughout this process, the solution remained clear and phase separation did not occur. Nitrogen was bubbled through the microemulsion for 15 minutes in order to remove any dissolved air. The microemulsion was then immediately irradiated using gamma-ray from a ¹³⁷Cs source (600 rad/min). Gamma-ray irradiation was used to initiate polymerization because thermal initiators have a tendency to destabilize the system. After irradiation for a period of 20 minutes, the flask was removed from the radiation chamber and the content was precipitated in large excess of acetone. The precipitate was then dispersed in water. In order to remove the unreacted amino acid, the dispersion was dialyzed at 25° C. for 24 h using a membrane with a molecular weight cut-off range of 1000 g/mol. The dialyzed product was lyophilized to remove the water. A similar procedure was followed for synthesizing chiral nanoparticles with 3, 6.5 and 8 mol % of D and L lysine. The size of the nanoparticles was determined by SEM analysis. SEM images were taken using a SEM 6335F instrument. The samples were dispersed in methanol and then dried on a copper wafer in vacuum. FIG. 13 shows that the particles are spherical, 100 nm in size, and monodispersed.

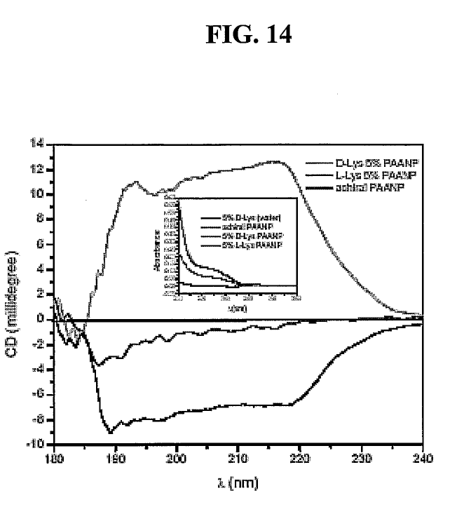

CD spectroscopy: The nano-sized building blocks obtained from poly(acrylic acid) containing chiral amino acid lysine have been shown to assemble into macromolecular chiral architectures with a helical sense that corresponds to the chiral center of the amino acid. The chiral nanoparticles generate CD spectra, the sign of which is consistent with the absolute configuration at the chiral carbon of the monoamine. As shown in FIG. 14, the CD spectra indicate a negative CD band for the L-lysine nanoparticle and a positive band for its enantiomer. The results were reproducible at different lysine concentrations. Although small variations were observed in the amplitudes of each enantiomer. UV spectroscopy (FIG. 14) demonstrates hyperchromicity of the chiral nanoparticles between 220 nm and 240 nm as compared to the PAANP without lysine, supporting the structural model of this invention that lysine is bound to PAANP. Furthermore, as compared to achiral PAANP, the CD signal is also enhanced between 187 nm and 240 nm.

Chiral nanoparticles obtained from poly(acrylic acid) and D- and L-lysine were prepared by reverse microemulsion technique. A chiroptical signal of the particles indicate that the copolymerized lysine causes the formation of chiral polymers with a conformation that corresponds to the absolute configuration of lysine. These nanoparticles are useful to encapsulate or extract optically active drugs, flavors and fragrances from their recemic mixture.

### EXAMPLE 7

### Extraction of Drugs at 37 Degrees

Extraction of drugs can also be performed at 37° C. (body temperature). At 37° C. and pH 7, poly(acrylic acid) nanoparticles extracted 93% of amitriptyline and 88% bupivacaine in 5 min. Under similar conditions in the presence of normal saline, poly(acrylic acid) nanoparticles extracted 87 and 77% of amitriptyline and bupivacaine respectively.

### EXAMPLE 8

The invention provides synthesis of novel polymeric nanoparticles for extraction of overdosed drugs and fragrances. We have synthesized poly(acrylamide) nanoparticles (50-100 nm) by polymerization of acrylamide in the presence of N,N′-methylene bisacrylamide as the crosslinker. Polymerization took place inside the micro-emulsion of AOT/toluene/water by g irradiation. The polyacrylamide nanoparticles, after modification with charged and hydrophobic groups, showed overdosed drug (amitriptyline) extraction of 80% compared to 18% for the unmodified nanoparticles. Nuclear Magnetic Resonance, Dynamic and Static Light Scattering, Fluorescence Spectroscopy, Surface Plasmon Resonance Spectroscopy and Ultra Violet Spectroscopy were used to characterize the synthesized nanoparticles and to monitor the extraction and release profile.

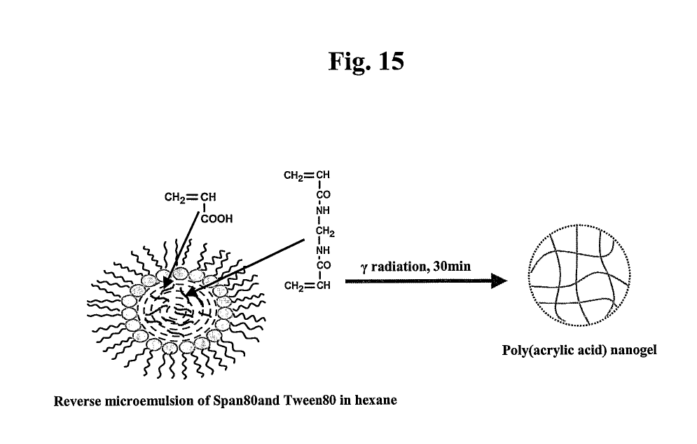

Synthesis and characterization of poly(acrylic acid) nanoparticles: Poly(acrylic acid) is pH sensitive, bioadhesive, biocompatible and biodegradable polymer. Porous poly(acrylic acid) nanoparticles were synthesized for extraction of overdosed drugs. Poly(acrylic acid) (PAA) nanoparticles with narrow size distribution were synthesized by inverse microemulsion polymerization technique. Polymerization took place inside the micro-emulsion of Span 80/Tween 80/hexane/water using gamma irradiation as shown in FIG. 15. N,N′-methylene bisacrylamide was used as the crosslinker.

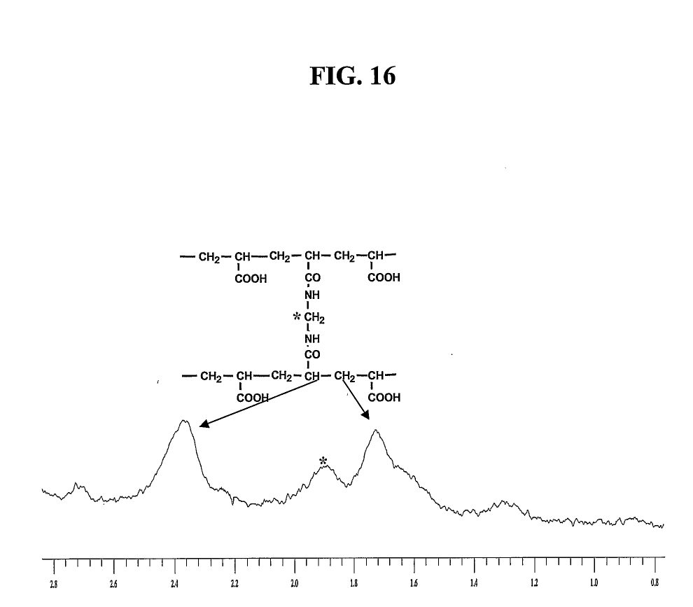

Hydrophobic modifications were carried out on nanoparticle system by incorporating N-acryloxysuccinimide into poly(acrylic acid) structure by copolymerization, followed by using the activity of the succinimide to substitute various chemical functions into the nanoparticle structure. This method allowed the introduction of hydrophobic hexyl groups and propyl groups. ¹H-NMR spectroscopy of poly(acrylic acid) nanoparticles in D₂O is shown in FIG. 16. In the product the methane protons of poly(acrylic acid) and N,N′-methylene-bis-acrylamide appeared at 2.4 ppm and the methylene protons appeared at 1.7 ppm. The methylene protons of the cross-linker(*) is visible at 1.9 ppm.

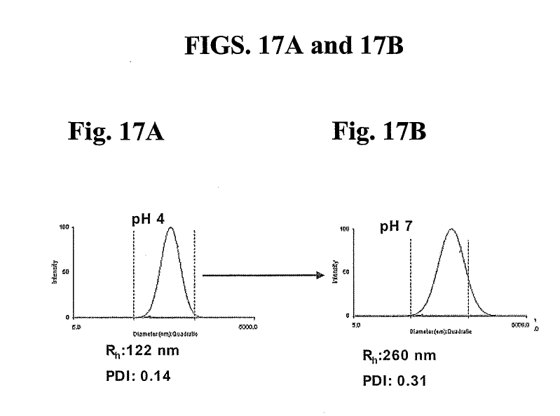

Scanning electron micrograph revealed that the particle size ranged from 50 nm to 80 nm and they are spherical in nature. Since the particles are very small, they can be well dispersed in the cosmetic and other chemical formulations. In case they are administered in human blood stream, these submicron size particles will avoid detection by the reticuloendothelial system. Hence can stay in the blood stream for a prolong time to extract overdosed drugs. Effective hydrodynamic radius of the particles (FIGS. 17A and 17B), as determined by Dynamic Light Scattering analysis showed that under neutral and alkaline conditions, poly(acrylic acid) nanoparticles swelled almost four times of their original dimension whereas in acidic condition swelling was not significant. Such swelling/shrinking is important for effective extraction and release of active agents.

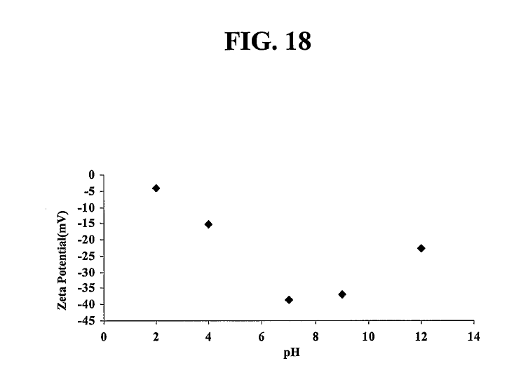

Surface charge studied by zeta potential measurement—Zeta potential measurement showed that the poly(acrylic acid) nanoparticles have negative zeta potential on their surfaces owing to the presence of anionic carboxylate ions. The negative value of the zeta potential increased with increase in the pH of the dispersion medium from 2 to 7. Further increase of pH of the dispersion medium from 7 to 12 decreased the negative zeta potential value due to the shielding of some of the carboxylate ions by added NaOH. The trend is shown in FIG. 18. This observation reveals the interaction of these nanoparticles with cationic attributes will be pH dependent.

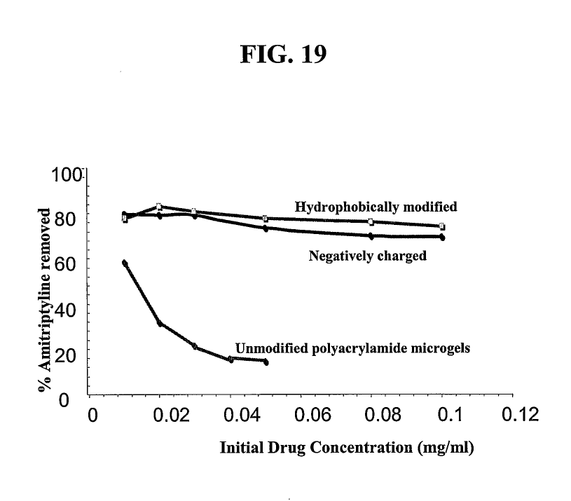

Extraction of drugs and fragrances by poly(acrylamide) nanoparticles: The invention provides for the synthesis of poly(acrylamide) nanoparticles. As shown in FIG. 19, modified poly(acrylamide) nanoparticles could extract approximately 80% of amitriptyline compared to 18% by the unmodified nanoparticles. Binding interaction between the nanoparticles and the drug depends on the functionality of the nanoparticle and the drug. Since the unmodified particle has a relatively neutral polymer backbone, the binding between the polymer backbone and cationic amitriptyline is minimal. In the case of the negatively charged nanoparticles, extraction is higher due to the electrostatic interaction with the positively charged drug molecules. Hydrophobic interaction between the drug and the hydrophobic moiety of the nanoparticles also results in the enhanced extraction of drug by hexylamine-modified nanoparticles.

Extraction of fragrance, linalyl acetate by poly(acrylic acid) nanoparticles: Linalyl acetate, one of the extensively used fragrance ingredients was incorporated into the nanoparticles by dispersing them in methanol followed by addition of linalyl acetate. It was observed that poly(acrylic acid) nanoparticles could extract 40% linalyl acetate(LA) from dispersion medium. The increased efficiency of extraction is accounted for by the incorporation of hydrophobic moieties along the polymer backbone.

pH dependent release of linalyl acetate from modified and unmodified nanoparticles: Release profile of linalyl acetate from the nanoparticles was pH dependent. More fragrance got released at neutral and alkaline pH than in acidic condition due to increased swelling of the nanoparticles at neutral and acidic pH. Furthermore, propylamine modified nanoparticles released the least amount of entrapped fragrance as compared to hexyl amine modified and unmodified nanoparticles.

Extraction of drugs by poly(acrylic acid) nanoparticles: The drugs under investigation were bupivacaine and amitriptyline. These drugs are responsible for cardiotoxicity if consumed in excess quantities. It was observed that extraction was pH dependant. Poly(acrylic acid) nanoparticles could extract around 60% of bupivacaine and amitriptyline in 4 hours at pH 7 and pH 9 whereas the extraction was negligible at pH 4. Higher extraction at neutral and alkaline pH is attributed to the enhanced swelling of the nanoparticles at pH 7 and 9.

Synthesis of chiral nanoparticles: Chiral recognition has attracted much attention due to its importance in the fields of biochemistry and biopharmacology. Chiral recognition by the nanoparticles is useful to allow selective extraction of an enantio-pure molecule from a recemic mixture of drugs, fragrances or other attributes. In order to induce optical activity on achiral poly(acrylic acid) nanoparticles, acrylic acid was copolymerized along with L-glutamic acid in the presence of the cross-linker inside the reverse microemulsion. Optical activity of the resultant product was detected by circular dichroism (CD) analysis.

Synthesis of starch nanoparticles: Starch microspheres are generally prepared by using the water-in-oil emulsion technique in the presence of epichlorohydrin as the cross-linking agent, which is carcinogenic. The invention provides, instead, for synthesis of starch nanoparticles using di-acids as the crosslinking agents. The di-acids are adipic acid, succinic acid, maleic acid and glutaric acid. These nanoparticles have a different extent of hydrophobicity and porosity, depending on the chain length of the cross-linker. Their rheological properties also vary.

In situ real time measurements of the responses of relevant molecules to physico-chemical changes in their environment were done. A surface plasmon resonance spectroscope was built in this work that uses the angle-scan method in a converging beam configuration. A “p”-polarized laser beam (632.8 nm) was focused on a prism metal interface to launch the surface plasmons. The reflected beam was collimated and captured using a CCD camera. SPR was used to explore the interfacial dynamics of the adsorbed surface-active species, particularly polymers when subjected to external perturbations. For studies on the conformational changes of polyacrylic acid (PAA), the overlayer solution pH was alternated between 3.5 and 9.5, and the stretching-coiling phenomenon thus induced was monitored in real time. The temporal profile of the pH change experiments showed the appearance of an inflexion point with increase in the molecular weight of the polyacrylic acid.

For investigating the polymer-surfactant interactions, polyacrylic acid (PAA) was immobilized on the SPR sensor surface and then exposed to dodecyl trimethyl ammonium chloride (DTAC). In an effort to control the loop size of the polymer upon adsorption, polyacrylic acid was thiolated to varying degrees: greater loop size implying a greater number of charge centers for the surfactants to attach to. Increased surfactant binding was observed with increase in the polymer loop size, which has thus opened up another degree of freedom in fine-tuning interfacial process phenomena. Binding of DTAC to PAA was discovered to take place in three distinct stages with the third step showing an increased rate over the second one. This increase in the rate was proposed to signify the sudden opening up of the polymeric structure. The formation of charged double surfactant species and the ensuing electrostatic repulsion was thought to be the factor that caused the polymer matrix to open up, rather than the natural tendency to collapse to form hydrophobic microdomains.

In an embodiment of the invention, polymers like poly(saccharides), poly(acrylic acid), poly(acrylamide), poly(acrylates) can be used to prepare the nanoparticles. The choice of polymer will depend on the type of applications required. Chiral amino acids such as L-glutamic acid and L-lysine will be used to induce chirality in the poly(acrylic acid) nanoparticles. Amine functionalized aptamers and allosteric DNAzyme crosslinkers will be used for synthesizing aptamer based nanoparticles.

Surfactants: Surfactants like Aerosol-OT (AOT), Sorbitan monooleate (Span 80), Polyoxyethylene (20) sorbitan monooleate (Tween 80) can be used to generate reverse microemulsion inside which the reactions will take place.

Nuclear Magnetic Resonance (NMR): This technique can be used to characterize the product formed by the reverse microemulsion method. NMR analysis will show the coupling between the crosslinkers and the polymeric backbone to produce a rigid network.

Scanning Electron Micrograph (SEM): SEM can reveal the size and the shape of the nanoparticles in dry state.

Dynamic Light Scattering (DLS): Effective hydrodynamic radius of the particles can be determined by DLS analysis. Effect of pH, temperature, and ionic strength on the hydrodynamic radius can be measured.

Spectroscopy: Fluorescence spectroscopy analysis can be performed on the hydrophobically modified nanoparticles to determine the extent of hydrophobicity in the system. Surface Plasmon Resonance spectroscopy can measure the kinetics of interaction between the nanoparticles and the actives.

High Pressure Liquid Chromatography (HPLC): Extraction and release of attributes from the nanoparticles in different solvents and buffers can be monitored by using HPLC.

Surface Plasmon Resonance (SPR): This technique can be used to study the kinetics of extraction of the attributes by the nanoparticles.

### EXAMPLE 9

### Nanoparticles to Fight Bioterrorism

Nanoparticles refer to a type of spherical, covalently cross-linked polymeric networks with a particle size in the nanometer range. Since nanogels are small in size with a porous structure, and an ability to be functionalized, they can act as potential scavengers/carriers for toxins and organics as well as sensor. As the fear and predictions of attacks with biological weapons are increasing, we envision the use of these smart nanoparticles as weapon against bio-terrorism due to the following properties of these particles: (1) ability to rapidly entrap desired molecules or bioagents; (2) ability to release at controlled rate; (3) small size of the particles for better dispersion; (4) reasonable shelf life and relatively stable under various storage and transport conditions.

The nanoparticles, as weapon against bio terrorism: (1) sense the changes in environmental conditions (2) process the sensed information (3) respond chemically or mechanically to the stimulus (4) have the ability for robust function under extreme conditions and (5) be cost-effective to manufacture.

Poly (acrylamide) (PAM) and Poly(acrylic acid) (PAA) nanoparticles (10-100 nm) with narrow size distribution were synthesized by inverse microemulsion polymerization technique. In order to prepare hydrophobic nanoparticles, N-acryloxysuccinimide was copolymerized into PAM and PAA structure, followed by substitution of succinimide by hydrophobic hexyl groups & propyl groups. The potential of PAA and hydrophobically modified PAA to extract and release amitriptyline was studied. To incorporate amitriptyline in PAA, the nanoparticles were dispersed in the amitriptyline solution in water. The efficacy of extraction enhanced when the unmodified nanoparticles were replaced by the hydrophobically modified nanoparticles. When the release profile of incorporated amitriptyline was monitored as a function of pH of the dispersion media, it was observed that the release was pH dependent. The ability of poly(acrylamide) and modified poly(acrylamide) nanoparticles to extract bupivacaine was investigated. It was observed that by using the modified nanoparticles the efficiency of extraction increased markedly from 18% to 80%. The kinetics of extraction was studied using Surface Plasmon Resonance (SPR) technique.

With the rising awareness of the public vulnerability to chemical and biological terrorism, there is a heightened need for detection and scavenging techniques that show both high sensitivity and selectivity. Such techniques also would find wide use in medicare diagnostics and fast drug delivery applications. The presently used techniques are time consuming and require multi step procedures. The challenge is to incorporate selectivity offered by ligand/receptor interactions into a system that can be extremely sensitive, robust and versatile. Also, it is important to design nanosystems that could detect and extract very small quantities of agents in vast open areas, in water systems, and human blood circulatory system. At the same time, they must be capable of adapting to changing temperature and humidity levels as well as wind, dust and other environmental factors. The nanosystem must be able to capture, concentrate and measure the levels of the target toxins as well as provide a signal that is measurable, such as a digital or audio signal or a change in color. This could be extended to a number of different things related to homeland security, such as the biological and chemical warfare agents.

To meet the present need of the nanosystems for various applications the invention provides for nanoparticles that sense external perturbations and respond instantaneously in a controlled fashion. Selectivity can be induced in the nanoparticles by choosing appropriate monomers, different functional groups and specific ligands thus making it possible for the nanoparticles to include or exclude the other materials selectively and to make them sensitive to various types of stimuli.

The synthesized nanoparticles can be modified as required and characterized in terms of their size, swelling behavior, charge, solubility, ionic strength, pH, temperature, polarity etc. Polymeric nanoparticles can be engineered in the following ways to fight against bio terrorism

1. Incorporate anionic fluorescence tag in the nanoparticles, which is sensitive towards the oppositely charged ions/polyanions/particles. For example, the fluorescence of the nanoparticles can be quenched by formation of relatively weak ground-state “donor-acceptor” complexes. Ligand concentration could then be estimated from the fluorescence intensity. The fluorescence tagged nanoparticles have sensing capabilities beyond ionic species or solutions.

2. Attach biosensors such as antigens and/or oligonucleotides to the surface of the nanoparticles, which is capable of responding to toxic malignant bio-agents. The interaction between antigen/antibody is detectable by the subsequent colorimetric change or fluorescence signal.

3. Administer biodegradable, biocompatible nanoparticles with suitable functional groups in human system to scavenge toxic substances that the human body might potentially be exposed to during chemical/biological warfare.

A series of functional nanopaticles can be tested to measure the structure-property relationship for the functional nanoparticles for efficient use of the kinetics and dynamics of extraction/release process. Nanoparticles that can be assembled into different patterns for extraction and sensing are encompassed by the invention and they can be characterized and modified as required. The sensing mechanism involves change in luminescence, electrochemical potential or current change, or a combination of luminescence and electrochemistry when the receptor molecules interact with the target molecules. These receptor molecules can be immobilized on the nanoparticles to obtain sensor and extraction arrays.

What is claimed is:

1. A method of preparing polymeric nanoparticles, comprising:
(a) solubilizing one or more nonionic and ionic surfactants, or a combination thereof, in organic solvent;
(b) introducing at least one polymerizing reagent to the solution of step (a) to form a reaction mixture;
(c) purging oxygen from the reaction mixture of step (b);
(d) exposing the purged reaction mixture of step (c) to gamma (γ) radiation for a time sufficient to crosslink nanoparticles formed in the reaction mixture; and
(e) precipitating the crosslinked nanoparticles from the reaction mixture.

2. The method according to claim 1, wherein the organic solvent comprises from 1-8 carbon atoms.

3. The method according to claim 2, wherein the organic solvent is hexane.

4. The method according to claim 1, wherein the at least one polymerizing reagent comprises one or more of acrylic acid and bisacrylamide monomers.

5. The method according to claim 1, wherein the surfactant is selected from sorbitan monooleate (SPAN 80®), polyoxyethylene(20) sorbitan monooleate (TWEEN 80%) or sodium bis 2-ethylhexyl sulfosuccinate (AOT).

6. The method according to claim 5, wherein the surfactant is selected from sorbitan monooleate (SPAN 80®) or polyoxyethylene(20) sorbitan monooleate (TWEEN 80®).

7. The method according to claim 1, wherein the introducing step (b) further comprises a coupling or linking agent.

8. The method according to claim 7, wherein the coupling or linking agent is N-acryloxysuccinimide.

9. The method according to claim 1, wherein the purging step (c) comprises passing the reaction mixture through nitrogen gas.

10. The method according to claim 1, wherein the nanoparticles are precipitated with acetone.

11. The method according to claim 1, further comprising washing the nanoparticles with organic solvent following step (e).

12. The method according to claim 11, wherein the organic solvent is hexane.

13. The method according to claim 7, further comprising the step of modifying the nanoparticles with a functional group.

14. The method according to claim 13, wherein the nanoparticles are modified by incorporation of a functional group selected from one or more hydrophobic groups, hydrophilic groups, enzymes, ionic groups, or a combination thereof.

15. The method according to claim 14, wherein the functional group comprises one or more hydrophobic groups.

16. The method according to claim 15, wherein the functional group comprises one or more alkyl amine groups.

17. The method according to claim 16, wherein the one or more alkyl amine groups comprise propylamine or hexylamine.

18. The method according to claim 15, further comprising incorporating a fragrance molecule into the hydrophobically modified nanoparticles.

19. The method according to claim 18, wherein the fragrance molecule is linalyl acetate or vanillin.

20. A method of releasing a fragrance, comprising:
(a) hydrophobically modifying a polymeric nanoparticle;
(b) incorporating a fragrance molecule within the hydrophobically modified nanoparticle; and
(c) releasing the fragrance from the nanoparticle.

21. The method according to claim 20, wherein the releasing step (c) involves one or more of (i) changing the crosslinking density of the nanoparticle; or (ii) changing the pH of the dispersion medium.

22. The method according to claim 20, further comprising the step of further modifying the nanoparticle with light sensitive molecules so that the fragrance is released upon exposure of the nanoparticle to light.

23. The method according to claim 22, wherein the light sensitive molecules are photolabile molecules.

24. The method according to claim 22, wherein the light is ultraviolet (UV) or non-UV light.

25. The method according to claim 20, further comprising co-polymerizing the hydrophobically modified nanoparticles with temperature-sensitive monomers to obtain temperature-sensitive nanoparticles comprising temperature-sensitive releasing properties of release of the fragrance.

26. The method according to claim 20, wherein the fragrance is linalyl acetate or vanillin.

27. The method according to claim 20, wherein in step (a) the nanoparticle is hydrophobically modified by the addition of N-acryloxysuccinimide to one or more polymerizing reagent monomers used to prepare modified nanoparticles.

28. The method according to claim 27, wherein the one or more polymerizing reagent monomers comprise acrylic acid and bisacrylamide monomers.

29. A method of releasing a biologically active molecule, comprising:
(a) hydrophobically modifying a polymeric nanoparticle;
(b) incorporating a biologically active molecule within the hydrophobically modified nanoparticle; and
(c) releasing the biologically active molecule from the nanoparticle.

30. The method according to claim 29, wherein the releasing step (c) involves one or more of (i) changing the crosslinking density of the nanoparticle; or (ii) changing the pH of the dispersion medium.

31. The method according to claim 29, further comprising co-polymerizing the hydrophobically modified nanoparticles with temperature-sensitive monomers to obtain temperature-sensitive nanoparticles comprising temperature-sensitive properties of release of the biologically active molecule.

32. The method according to claim 29, further comprising the step of further modifying the nanoparticle with light sensitive molecules so that the fragrance is released upon exposure of the nanoparticle to light.

33. The method according to claim 32, wherein the light sensitive molecules are photolabile molecules.

34. The method according to claim 32, wherein the light is ultraviolet (UV) or non-UV light.

35. The method according to claim 29, wherein the biologically active molecule is selected from one or more of drugs, small molecules, antimicrobial agents, antibiotics, antitoxins, antibodies, pesticides, biocides, detoxifying agents, antifungal agents, enzymes, proteins, RNA molecules, antisense molecules, or a combination thereof.

36. The method according to claim 29, wherein in step (a) the nanoparticle is hydrophobically modified by the addition of N-acryloxysuccinimide to one or more polymerizing reagent monomers used to prepare the nanoparticles.

37. The method according to claim 36, wherein the one or more polymerizing reagent monomers comprise acrylic acid and bisacrylamide monomers.

38. A method of preparing a functionally modified polymeric nanoparticle, comprising:
(a) solubilizing one or more nonionic and ionic surfactants, or a combination thereof, in organic solvent;
(b) introducing (i) at least one polymerizing monomer reagent and (ii) a linking reagent to the solution of step (a) to form a reaction mixture;
(c) purging oxygen from the reaction mixture of step (b);
(d) exposing the purged reaction mixture of step (c) to gamma (γ) radiation for a time sufficient to crosslink nanoparticles formed in the reaction mixture;
(e) introducing a functional group or molecule into the reaction mixture; and
(f) precipitating the crosslinked and functionally modified nanoparticles from the reaction mixture.

39. The method according to claim 38, wherein the organic solvent comprises from 1-8 carbon atoms.

40. The method according to claim 38, wherein the organic solvent is hexane.

41. The method according to claim 38, wherein the at least one polymerizing monomer reagent comprises acrylic acid and bisacrylamide monomers.

42. The method according to claim 38, wherein the surfactant is selected from sorbitan monooleate (SPAN 80®), polyoxyethylene(20) sorbitan monooleate (TWEEN 80®) or sodium bis 2-ethylhexyl sulfosuccinate (AOT).

43. The method according to claim 42, wherein the surfactant is selected from sorbitan monooleate (SPAN 80®) or polyoxyethylene(20) sorbitan monooleate (TWEEN 80®).

44. The method according to claim 38, wherein the purging step (c) comprises passing the reaction mixture through nitrogen gas.

45. The method according to claim 38, wherein the nanoparticles are precipitated with acetone.

46. The method according to claim 38, further comprising washing the nanoparticles with organic solvent following step (d).

47. The method according to claim 46, wherein the organic solvent is hexane.

48. The method according to claim 38, wherein the nanoparticles are functionally modified by incorporation of a functional group or molecule selected from one or more hydrophobic groups or molecules, hydrophilic groups or molecules, enzymes, magnetic groups or molecules, or ionic groups or molecules.

49. The method according to claim 48, wherein the functional group or molecule comprises one or more hydrophobic groups.

50. The method according to claim 49, wherein the functional group comprises one or more alkylamine groups.

51. The method according to claim 50, wherein the one or more alkyl amine groups comprise propylamine or hexylamine.

52. The method according to claim 38, further comprising incorporating or encapsulating a fragrance molecule into the functionally modified nanoparticles.

53. The method according to claim 52, wherein the fragrance molecule is selected from linalyl acetate or vanillin.

54. The method according to claim 38, further comprising incorporating or encapsulating a biologically active molecule into the functionally modified nanoparticles.

55. The method according to claim 54, wherein the biologically active molecule is selected from one or more of drugs, small molecules, antimicrobial agents, antibiotics, antitoxins, antibodies, pesticides, biocides, detoxifying agents, antifungal agents, enzymes, proteins, RNA molecules, antisense molecules, or a combination thereof.

56. The method according to claim 35 or claim 55, wherein the biologically active molecule is a drug.

57. The method according to claim 56, wherein the drug is bupivacaine or amitriptyline.

58. A method of preparing hydrophobically modified polymeric nanoparticles, comprising:
(a) solubilizing one or more nonionic and ionic surfactants, or a combination thereof, in organic solvent;
(b) introducing (i) at least one polymerizing monomer reagent and (ii) a linking reagent to the solution of step (a) to form a reaction mixture;
(c) purging oxygen from the reaction mixture of step (b);
(d) exposing the purged reaction mixture of step (c) to gamma (γ) radiation for a time sufficient to crosslink nanoparticles formed in the reaction mixture;
(e) introducing a hydrophobic functional group or molecule into the reaction mixture; and
(f) precipitating the crosslinked and hydrophobically modified nanoparticles from the reaction mixture.

59. The method according to claim 58, wherein the organic solvent comprises from 1-8 carbon atoms.

60. The method according to claim 58, wherein the organic solvent is hexane.

61. The method according to claim 58, wherein the at least one polymerizing monomer reagent comprises acrylic acid and bisacrylamide monomers.

62. The method according to 58, wherein the surfactant is selected from sorbitan monooleate (SPAN 80®), polyoxyethylene(20) sorbitan monooleate (TWEEN 80®) or sodium bis 2-ethylhexyl sulfosuccinate (AOT).

63. The method according to claim 62, wherein the surfactant is selected from sorbitan monooleate (SPAN 80®) or polyoxyethylene(20) sorbitan monooleate (TWEEN 80®).

64. The method according to claim 58, wherein the purging step (c) comprises passing the reaction mixture through nitrogen gas.

65. The method according to claim 58, wherein the nanoparticles are precipitated with acetone.

66. The method according to claim 58, further comprising washing the nanoparticles with organic solvent following step (d).

67. The method according to claim 66, wherein the organic solvent is hexane.

68. The method according to claim 58, wherein the hydrophobic functional group or molecule comprises one or more alkylamine groups.

69. The method according to claim 68, wherein the one or more alkylamine groups comprise propylamine or hexylamine.

70. The method according to claim 58, further comprising incorporating or encapsulating a fragrance molecule into the hydrophobically modified nanoparticles.

71. The method according to claim 70, wherein the fragrance molecule is selected from linalyl acetate or vanillin.

72. The method according to claim 58, further comprising incorporating or encapsulating a biologically active molecule into the functionally modified nanoparticles.

73. The method according to claim 72, wherein the biologically active molecule is selected from one or more of drugs, small molecules, antimicrobial agents, antibiotics, antitoxins, antibodies, pesticides, biocides, detoxifying agents, antifungal agents, enzymes, proteins, RNA molecules, antisense molecules, or a combination thereof.

74. The method according to claim 73, wherein the biologically active molecule is a drug.

75. The method according to claim 74, wherein the drug is bupivacaine or amitriptyline.
